(19) United States
(12) Patent Application Publication
(10) Pub. No.: US 2024/0105318 A1
(43) Pub. Date: Mar. 28, 2024

(54) MEDICAL FACILITY MATCHING SYSTEM, MEDICAL FACILITY MATCHING METHOD, AND RECORDING MEDIUM

(71) Applicant: NEC Corporation, Minato-ku, TOKYO (JP)

(72) Inventors: Yuan LUO, Tokyo (JP); Kosuka NISHIHARA, Tokyo (JP); Masahiro HAYASHITANI, Tokyo (JP)

(73) Assignee: NEC Corporation, Minato-ku, TOKYO (JP)

(21) Appl. No.: 18/266,983

(22) PCT Filed: Dec. 21, 2020
(86) PCT No.: PCT/JP2020/047662

§ 371 Date: Jun. 13, 2023

(51) Int. Cl. G16H 40/20

(52) U.S. Cl. CPC G16H 40/20

(57) ABSTRACT

A medical facility matching system (1) comprises: a matching processing unit (121) performing matching processing to select as a matching result, a recommended medical facility for a patient based on patient item information (D12) corresponding to patient attribute items (Di12) and facility item information (D22) corresponding to facility attribute items (Di22); a data learning unit (122) learning about the patient attribute items for the matching processing so that the matching result is likely to be approved by the patient, and learning about the facility attribute items for the matching processing so that the matching result is likely to be approved by the medical facility selected; and a data item choosing unit (123) choosing based on a learning result, the patient attribute items and facility attribute items for the matching processing, wherein the matching processing is performed based on the chosen patient attribute items and the chosen facility attribute items.

### TECHNICAL FIELD

The present disclosure relates to a medical facility matching system, a medical facility matching method, and a recording medium, each being for matching between a patient and a medical facility.

### BACKGROUND ART

As a system of this type, there is known a system in which a plurality of medical facilities suitable for a patient are selected based on information about the patient and information about medical facility, and a booking is executed for a medical facility determined by the patient from the medical facilities selected (for example, refer to Patent Literature 1).

### CITATION LIST

### Patent Literature

Patent Literature 1: JP-A-2007-048101

### SUMMARY

### Technical Problem

However, according to the technique disclosed in Patent Literature 1, merely disclosed is that: a degree of conformance is calculated by combining the information about the patient with the information about each medical facility; and when the degree of conformance is equal to or greater than a predetermined criterion, the medical facility for the patient is selected. There is no description that one selection result is utilized in another selection processing. Therefore, it may not be possible to make efficient medical facility selection processing based on previous selection results.

The present disclosure shows a medical facility matching system, a medical facility matching method, and a recording medium, each making the selection processing of medical facility efficient.

### Solution to Problem

An aspect of a medical facility matching system according to the present disclosure includes a medical facility matching system comprising: a matching processing unit that is configured to perform matching processing to select as a matching result, at least one medical facility to be recommended for a patient from a plurality of medical facilities, based on patient item information corresponding to a plurality of patient attribute items with respect to the patient and facility item information corresponding to a plurality of facility attribute items with respect to each of the plurality of medical facilities; a data learning unit that is configured to learn about the plurality of patient attribute items necessary for the matching processing so that the matching result is likely to be approved by the patient, and learn about the plurality of facility attribute items necessary for the matching processing so that the matching result is likely to be approved by the medical facility selected; and a data item choosing unit that is configured to choose based on a learning result of the data learning unit, the plurality of patient attribute items to be used in the matching processing and the plurality of facility attribute items to be used in the matching processing, wherein the matching processing unit is configured to perform the matching processing based on the patient item information corresponding to the plurality of patient attribute items chosen and the facility item information corresponding to the plurality of facility attribute items chosen.

An aspect of a medical facility matching method according to the present disclosure includes a medical facility matching method of making a computer: perform matching processing to select as a matching result, at least one medical facility to be recommended for a patient from a plurality of medical facilities, based on patient item information corresponding to a plurality of patient attribute items with respect to the patient and facility item information corresponding to a plurality of facility attribute items with respect to each of the plurality of medical facilities; learn about the plurality of patient attribute items necessary for the matching processing so that the matching result is likely to be approved by the patient, and learn about the plurality of facility attribute items necessary for the matching processing so that the matching result is likely to be approved by the medical facility selected; choose based on a learning result with respect to the plurality of patient attribute items and the plurality of facility attribute items, the plurality of patient attribute items to be used in the matching processing and the plurality of facility attribute items to be used in the matching processing; and perform the matching processing based on the patient item information corresponding to the plurality of patient attribute items chosen and the facility item information corresponding to the plurality of facility attribute items chosen.

An aspect of a recording medium according to the present disclosure includes a recording medium on which a computer program is recorded, the computer program being configured to allow a computer to function as: a matching processing unit that is configured to perform matching processing to select as a matching result, at least one medical facility to be recommended for a patient from a plurality of medical facilities, based on patient item information corresponding to a plurality of patient attribute items with respect to the patient and facility item information corresponding to a plurality of facility attribute items with respect to each of the plurality of medical facilities; a data learning unit that is configured to learn about the plurality of patient attribute items necessary for the matching processing so that the matching result is likely to be approved by the patient, and learn about the plurality of facility attribute items necessary for the matching processing so that the matching result is likely to be approved by the medical facility selected; and a data item choosing unit that is configured to choose based on a learning result of the data learning unit, the plurality of patient attribute items to be used in the matching processing and the plurality of facility attribute items to be used in the matching processing, wherein the matching processing unit is configured to perform the matching processing based on the patient item information corresponding to the plurality of patient attribute items chosen and the facility item information corresponding to the plurality of facility attribute items chosen.

According to one aspect of each of medical facility matching system, medical facility matching methods, and recording medium described above, the selection processing of medical facility could be made more efficient.

### BRIEF DESCRIPTION OF DRAWINGS

FIG. 1 is a block diagram showing an exemplary entire configuration of medical facility matching system according to the present disclosure.

FIG. 2A is a diagram showing an example of patient matching data.

FIG. 2B is a diagram showing an example of a patient attribute item list.

FIG. 3A is a diagram showing an example of facility matching data.

FIG. 3B is a diagram showing an example of a facility attribute item list.

FIG. 4 is a block diagram showing an example of a configuration of a matching server.

FIG. 5 is a block diagram showing an example of a configuration of a patient-side terminal.

FIG. 6 is a block diagram showing an example of a configuration of a facility-side terminal.

FIG. 7 is a diagram showing an example of patient attribute items and facility attribute items that have been learned with respect to the medical examination for bowel cancer.

FIG. 8 is a diagram showing an example of patient attribute items and facility attribute items that have been learned with respect to the medical examination for cerebral infarction.

FIG. 9 is a diagram showing an example of patient attribute items and facility attribute items that have been learned with respect to the medical examination for cardiac infarction.

FIG. 10 is a flow chart showing an example of operation flow in the medical facility matching system.

### DESCRIPTION OF EXAMPLE EMBODIMENTS

### 1. Entire Configuration of Medical Facility Matching System

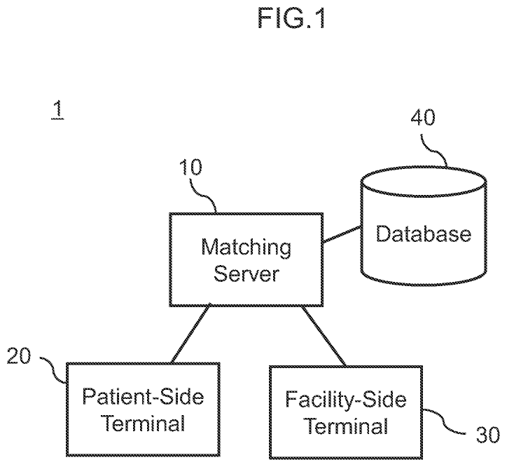

First, the entire configuration of medical facility matching system 1 (hereinafter, referred to as “matching system 1”) according to the present disclosure will be described. FIG. 1 is a block diagram showing an example of the matching system 1.

### 1.1 Matching System

The matching system 1 according to the present discloser is, for example, configured to: select (i.e., match) at least one medical facility to be recommended for a patient as a recommended facility when the patient plans to undergo medical examination (i.e., at least one of medical inspection and clinical care); obtain a patient-side approval result indicating approval or disapproval to the selection result (hereinafter referred to as the “matching result”) on the patient side and a facility-side approval result indicating approval or disapproval to the matching result on the medical facility side; and exploit these approval results for the selection of the recommended facility in the future.

The medical examination that a patient plans to undergo may include at least one of the following: a medical examination that the patient desires to undergo; a medical examination that the patient is recommended to undergo; and a medical examination that the patient should undergo. In the following, for convenience of explanation, the medical examination that a patient plans to undergo is referred to as the “recommended examination”. The “patient” in the present disclosure is a person who is an applicable target to the matching system 1, the person planning to undergo the recommended examination. The “patient” is, for example, not necessary to be in treatment for any of diseases. The “patient” may include, for example, a patient who has ever undergone any medical examination in a medical facility, the medical facility being an applicable target to the matching system 1. The patient may also include a patient who was recommended to receive a recommended examination at a certain medical facility. The “medical facility” is a facility that can provide at least one medical examination for any kind of diseases, the facility being the applicable target to the matching system 1. The medical facility also includes, for example, a medical facility where doctors do not stay at all times.

The range of the “applicable target to the matching system 1” may be set as appropriate depending on an organization, a structure, an environment, or the like as an installation target of the matching system 1. For example, the medical facilities and patients relating to a predetermined area may be set as the applicable targets to the matching system 1. Alternatively, the medical facilities and patients belonging to a predetermined organization may be set as the applicable targets to the matching system 1.

In the matching system 1, as shown in FIG. 1, a matching server 10 is provided. As illustrated in FIG. 1, in the matching system 1, for example, a patient-side terminal 20, a facility-side terminal 30, and a database 40 may be provided so as to communicate data to the matching server 10. The mode of each data communication may be network communication via a communication network or direct connection, regardless of wired or wireless. Though one patient-side terminal 20 and one facility-side terminal 30 are connected in FIG. 1, the patient-side terminals 20 corresponding to a plurality of patients respectively and the facility-side terminals 30 corresponding to a plurality of medical facilities respectively may be connected as appropriate to the matching server 10.

### 1.2 Patient-Side Terminal

The patient-side terminal 20 is an information processing apparatus capable of input/output processing of information relating to the patient, mainly under the control of the matching server 10. The patient terminal 20 performs, for example, processing relating to data input on the patient side, the data being used for matching, processing relating to response on the patient side, the response indicating approval or disapproval to the matching result, and the like. As such, the patient-side terminal 20 serves as a patient side interface in the matching system 1. An operator who operates the patient-side terminal 20, hereinafter referred to as “patient-side operator”. The patient-side operator may be the patient him/herself or may be a different person from the patient. The patient-side operator may be, for example, a person working for a medical facility. As the patient-side terminal 20, an information processing apparatus (for example, at least one of a personal computer, a tablet terminal, and a smartphone) possessed by the patient-side operator may be used. Further, as the patient-side terminal 20, an information processing apparatus (e.g., at least one of a personal computer and a tablet terminal) installed in a predetermined facility (e.g., a medical facility) may be used.

### 1.3 Facility-Side Terminal

The facility-side terminal 30 is an information processing apparatus capable of performing input/output processing of information of the medical facility side mainly under the control of the matching server 10. The facility-side terminal 30 performs, for example, processing relating to data input on the medical facility side, the data being used for matching and processing relating to response on the facility side, the response indicating approval or disapproval to the matching result. Thus, the facility-side terminal 30 functions as the facility side interface in the matching system 1. An operator who operates the facility-side terminal 30, hereinafter referred to as “facility-side operator”. The facility-side operator may be, for example, a person wording for a medical facility where the matching system 1 is installed or a person in charge of an operation facility (e.g., a public facility or government office) operating the matching system 1. An information processing apparatus (for example, at least one of a personal computer, a tablet terminal, and a smart phone) possessed by the facility-side operator may be used as the facility-side terminal 30. Further, as the facility-side terminal 30, an information processing apparatus (e.g., at least one of a personal computer and a tablet terminal) installed in a predetermined facility (e.g., a medical facility) may be used.

The patient-side operator and the facility-side operator may be the same person, or the patient-side terminal 20 and the facility-side terminal 30 may be physically one terminal.

### 1.4 Matching Server

The matching server 10 may be physically configured as a single server apparatus, or it may be configured as a cloud server virtually formed by a plurality of server apparatuses. The matching server 10 performs selection processing (hereinafter, referred to as “matching processing”) in which at least one recommended facility is selected for the patient based on data for matching corresponding to each patient (hereinafter, referred to as “patient matching data”) and data for matching corresponding to each medical facility (hereinafter, referred to as “facility matching data”).

### 1.5 Patient Matching Data

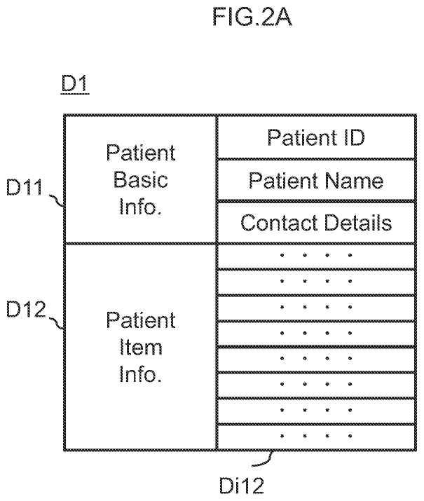

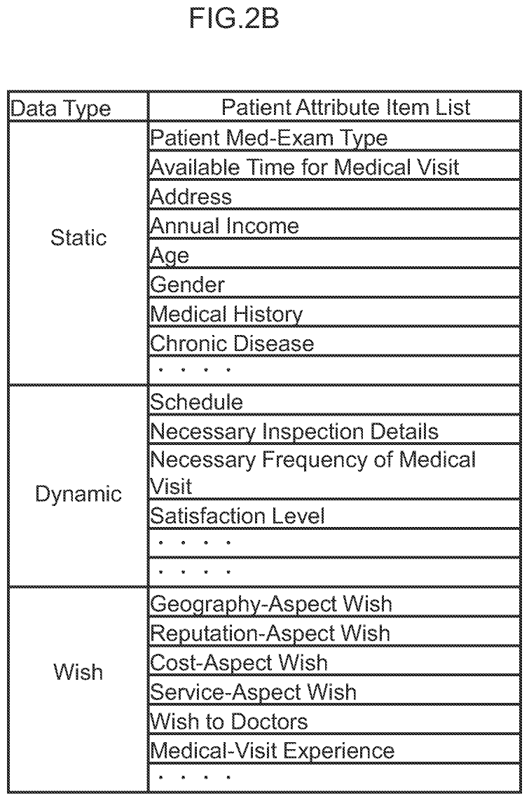

An example of patient matching data D1 which is used in the matching processing will be described referring to FIG. 2A and FIG. 2B. The patient matching data D1 is data generated for each patient. The patient matching data D1 includes, for example, patient basic information D11 and patient item information D12 as shown in FIG. 2A. The patient basic information D11 is basic information for specifying each patient. The patient basic information D11 includes, for example, patient identification information (i.e., the “patient ID”) for identifying each patient, the patient name, and the contact details (e.g., mail address). The patient item information D12 is information corresponding to a plurality of patient attribute items Di12 and is referred to as patient-side matching elements to be considered in matching. The plurality of patient attribute items Di12 are items relating to various attributes of the patient. The patient attribute items Di12 include, for example, at least a part of a plurality of data items included in the patient attribute item list shown in FIG. 2B. The plurality of patient attribute items Di12 included in the patient matching data D1 are formed by data items chosen from the patient attribute item list based on a learning result of data item learning processing, which will be described later.

The data items in the patient attribute item list may include, for example, static items, dynamic items, and wish items, as shown in FIG. 2B. The “static item” is a data item that has no change over time or that has less change over time than the dynamic item. The static items include, for example, patient med-exam type, available time for medical visit, address, annual income, age, gender, medical history, chronic disease, etc. The “patient med-exam type” indicates examination identification information for identifying each type of the recommended examination. The “dynamic item” is a data item that changes over time or is more likely to change over time than the static item. The dynamic items include, for example, schedule, necessary inspection details, and necessary frequency of medical visit, “satisfaction level”, etc. The “schedule” is the patient's behavior schedule, and may include, for example, available date and time for medical visit with respect to the patient. The “necessary inspection details” indicates the type(s) of inspection(s) required in the recommended examination. The “satisfaction level” indicates the degree of satisfaction with past medical examinations. The “wish item” is a data item relating to a request for medical facility from the patient. The wish item includes, for example, geography-aspect wish, reputation-aspect wish, cost-aspect wish, service-aspect wish, wish to doctors, medical-visit experience, and the like. The “geography-aspect wish” is a geographical wish for medical facilities, such as a wish for a geographical relationship (e.g., near, short walk distance, or the like) to his/her own home, or a wish for a location for medical facilities. The “reputation-aspect wish” is a wish with respect to reputation for medical facilities. The “cost-aspect wish” is a wish with respect to the cost of the recommended examination provided by medical facilities. The “service-aspect wish” is a wish with respect to a service which would be provided by medical facilities. The “service-aspect wish” may include, for example, preferred type and/or number of shops provided in medical facilities, preferred average waiting time, and preferred free service. Each of the static, dynamic and wish items may, for example, include a minimum data item necessary for the matching processing (e.g., a coincidence-required item which will be described below). Alternatively, for example, only the static item may include the minimum data item. With respect to the minimum data item, for example, in a case that the minimum data item is always chosen as the patient attribute item Di12, the data item learning which will be described below is not necessary. In addition, since the static items are unchangeable or unlikely to change, the weight thereof is large as the patient attribute. Thus, for example, for the respective static items, the priority (the weight) regarding the matching may be set larger compared to the dynamic items and the wish items.

### 1.6 Facility Matching Data

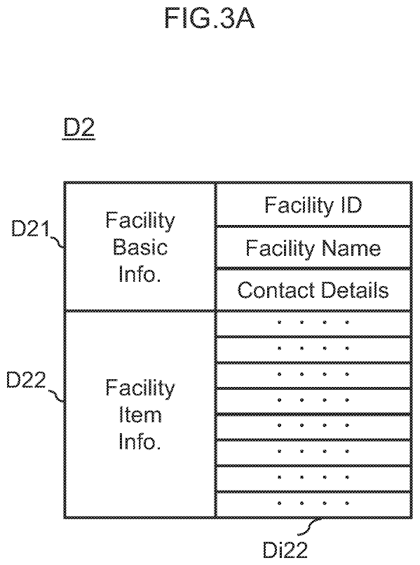

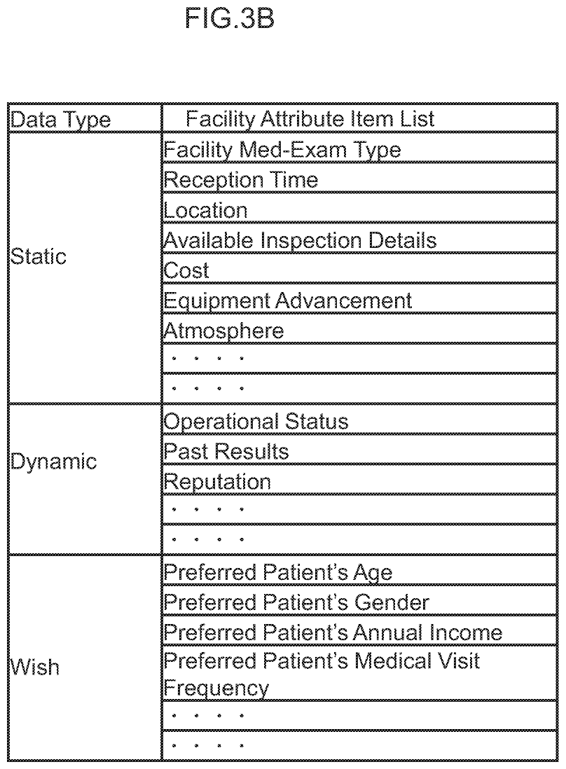

An example of the facility matching data D2 which is used in the matching processing will be described with reference to FIG. 3A and FIG. 3B. The facility matching data D2 is data generated for each medical facility. The facility matching data D2 includes a facility basic information D21 and a facility item information D22, for example, as shown in FIG. 3A. The facility basic information D21 is basic information for specifying each medical facility. The facility basic information D21 includes, for example, the facility identification information (i.e., the “facility ID”) for identifying each medical facility, the medical facility name, and the contact details (e.g., the mail address). The facility item information D22 is information corresponding to a plurality of facility attribute items Di22 and is referred to as facility-side matching elements to be considered in matching. The plurality of facility attribute items Di22 are items relating to various attributes of the medical facility. The facility attribute items Di22 includes, for example, at least a part of a plurality of data items included in the facility attribute item list shown in FIG. 3B. The plurality of facility attribute items Di22 included in the facility matching data D2 are formed by data items chosen from the facility attribute item list based on the learning result of the data item learning processing which will be described below.

The data items in the facility attribute item list may include, for example, static items, dynamic items, and wish items as shown in FIG. 3B. The “static item” is a data item that has no change over time or that has less change over time than the dynamic item. The static items include, for example, its facility med-exam type, reception time, location, available inspection details, cost, equipment advancement, atmosphere, etc. The “facility med-exam type” indicates examination identification information for identifying each type of medical examination that the medical facility can provide. The “available inspection details” indicates the type(s) of inspection(s) that can be provided for the corresponding medical examination. The “dynamic item” is a data item that changes over time or is more likely to change over time than the static item. The dynamic items include, for example, its operational status, past results, reputation, etc. The “operational status” indicates a booking status (i.e., a vacant status) of the corresponding medical examination. The “reputation” indicates a satisfaction level of patients who took in the past the same type of medical examination indicated as the “facility med-exam type”. The “wish item” is a data item relating to a request for patients from the medical facility. The wish item may be a data item relating to a request for patients from the medical facility in order for the medical facility to manage appropriately depending on the resource thereof. The wish items may include; for example, with respect to patients, age (preferred patient's age), gender (preferred patient's gender), annual income (preferred patient's annual income), necessary frequency of medical visit (preferred patient's medical-visit frequency); preferred medical examination or inspection corresponding to the facility med-exam type; etc. Each of the static, dynamic and wish items may, for example, include a minimum data item necessary for the matching processing (e.g., the coincidence-required item which will be described below). Alternatively, for example, only the static items may include the minimum data items. With respect to the minimum data item, for example, in a case that the minimum data item is always chosen as the facility attribute item Di22, the data item learning which will be described below is not necessary. In addition, since the static items are unchangeable or unlikely to change, the weight thereof is large as the facility attribute. Thus, for example, for the respective static items, the priority (weight) regarding the matching may be set larger compared to the dynamic items and the wish items.

### 1.7 Database

Referring back to FIG. 1, the database 40 stores various information to be used for the patient matching data D1 (i.e., patient relating information) and various information to be used for the facility matching data D2 (i.e., facility relating information). Each of the patient matching data D1 and facility matching data D2 may not be present in the database 40 in advance as data having the data structure described above. For example, the database 40 may include: electronic medical chart data (including electronic medical charts and/or data relating to electronic medical charts) of patients managed by the matching system 1; registered data of patients managed by at least one facility employing the matching system 1; evaluation data managed by at least one word-of-mouth communication site with respect to medical facilities. Further, at least a part of various kinds of information included in the patient matching data D1 and/or at least a part of various kinds of information included in the facility matching data D2 may be included in the database 40 as information which is utilized in at least one system different from the matching system 1 or as information managed by at least one database different from the database 40. Accordingly, the database 40 may include for example: medical chart data (including electronic medical charts and/or data relating to electronic medical charts) of patients managed by a medical system that differs from the matching system 1; registered data of patients managed by a facility (for example, a sports facility) that does not employ the matching system 1; evaluation data managed by a word-of mouth communication site with respect to medical facilities; and the like.

### 2. Configuration of the Matching Server

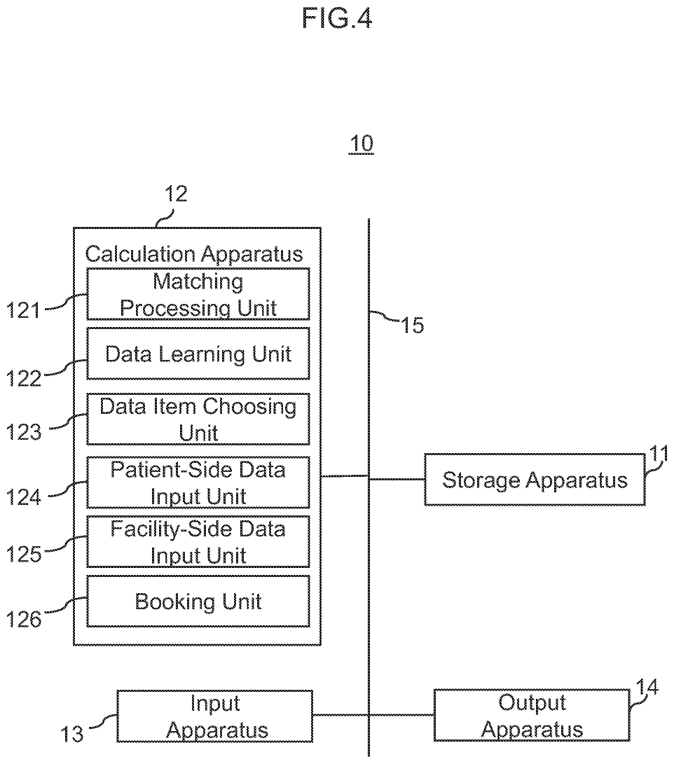

Subsequently, the configuration of the matching server 10 will be described with reference to FIG. 4. FIG. 4 is a block diagram illustrating an example of the configuration of the matching server 10. As shown in FIG. 4, the matching server 10 includes a storage apparatus 11 and a calculation apparatus 12. Further, the matching server 10 may include an input apparatus 13 and an output apparatus 14. However, the matching server 10 may not include at least one of the input apparatus 13 and the output apparatus 14. The storage apparatus 11, calculation apparatus 12, input apparatus 13, and output apparatus 14 may be connected to each other through a data bus 15.

The storage apparatus 11 is capable of storing desired data. For example, the storage apparatus 11 may temporarily store computer programs executed by the calculation apparatus 12. The storage apparatus 11 may temporarily store data used by the calculation apparatus 12 temporarily when the calculation apparatus 12 is executing a computer program. The storage apparatus 11 may store data to be held in long periods by the matching server 10 such as, for example, the patient attribute item list (FIG. 2B) and the facility attribute item list (FIG. 3B). The storage apparatus 11 may include at least one of RAM (Random Access Memory), ROM (Read Only Memory), a hard disk apparatus, a magneto-optical disk array apparatus, an SSD (Solid State Drive), and a disk array apparatus. In other words, the storage apparatus 11 may include a volatile recording medium and a non-volatile recording medium.

The calculation apparatus 12 includes, for example, CPU (Central Processing Unit). The calculation apparatus 12 reads computer programs. For example, the calculation apparatus 12 may read computer programs stored in the storage apparatus 11. For example, the calculation apparatus 12 may read computer programs stored in a non-volatile recording medium readable by a computer, using a not-shown recording medium read apparatus. The calculation apparatus 12 may acquire (that is, download or read) a computer program from a not-shown apparatus arranged outside the matching server 10 through a not-shown communication apparatus. The calculation apparatus 12 executes the loaded computer program. Consequently, in the calculation apparatus 12, logical function blocks for performing operations to be performed by the matching server 10 are realized. That is, the calculation apparatus 12 is capable of functioning as a controller for realizing the logical function blocks for performing operations to be performed by the matching server 10.

FIG. 4 shows an example of logical function blocks realized in the calculation apparatus 12 for performing the respective processing in the matching system 1. As shown in FIG. 4, in the calculation apparatus 12, a matching processing unit 121, a data learning unit 122, a data item choosing unit 123, a patient-side data input unit 124, a facility-side data input unit 125, and a booking unit 126 are realized. Details of the operations of each of the sections 121 to 126 will be described later.

The input apparatus 13 is an apparatus that accepts input of information to the matching server 10 from the outside of the matching server 10. For example, the input apparatus 13 may acquire (i.e., receive) various kinds of information from each of the patient-side terminal 20 and the facility-side terminal 30.

The output apparatus 14 is an apparatus that outputs information to the outside of the matching server 10. For example, the output apparatus 14 may output various information relating to each processing performed by the matching server 10. For example, the output apparatus 14 may output (i.e., transmit) various control data to each of the patient-side terminal 20 and the facility-side terminal 30.

### 3. Configuration of Patient-Side Terminal

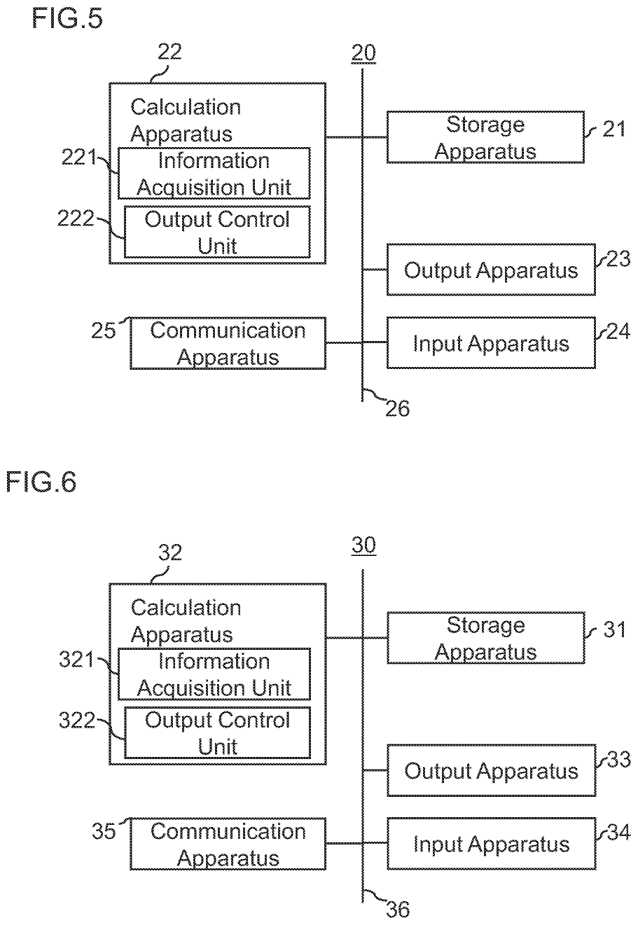

The configuration of the patient-side terminal 20 will be described with reference to FIG. 5. FIG. 5 is a block diagram showing an example of the composition of the patient-side terminal 20. As shown in FIG. 5, the patient-side terminal 20 includes a storage apparatus 21, a calculation apparatus 22, an output apparatus 23, an input apparatus 24, and a communication apparatus 25. The storage apparatus 21, calculation apparatus 22, output apparatus 23, input apparatus 24, and communication apparatus 25 may be connected to each other through a data bus 26.

The storage apparatus 21 is capable of storing desired data. For example, the storage apparatus 21 may temporarily store computer programs to be executed by the calculation apparatus 22. The storage apparatus 21 may temporarily store data that is temporarily used by the calculation apparatus 22 when the calculation apparatus 22 is executing a computer program. The storage apparatus 21 may store data to be held in long periods by the patient-side terminal 20. The storage apparatus 21 may include at least one of a RAM, ROM, a hard disk apparatus, a magneto-optical disk array apparatus, an SSD, and a disk array apparatus. In other words, the storage apparatus 21 may include a volatile recording medium and a non-volatile recording medium.

The calculation apparatus 22 includes, for example, a CPU. The calculation apparatus 22 reads computer programs. For example, the calculation apparatus 22 may read computer programs stored in the storage apparatus 21. For example, the calculation apparatus 22 may read a computer program stored in a computer readable non-volatile recording medium, using a not-shown recording medium read apparatus. The calculation apparatus 22 may acquire (i.e., download or read) a computer program from a not-shown apparatus arranged outside the patient-side terminal 20 via the communication apparatus 25. The calculation apparatus 22 executes the loaded computer program. Consequently, logical function blocks are realized in the calculation apparatus 22 for performing operations to be performed by the patient-side terminal 20. That is, the calculation apparatus 22 is capable of functioning as a controller for realizing the logical function blocks for performing operations to be performed by the patient-side terminal 20.

In the present disclosure, the patient-side terminal 20 performs: a patient-side data inputting operation for inputting to the patient-side terminal 20 under the control of the matching server 10, information required for each processing (for example, matching processing, approval processing, and the like) in the matching server 10; and a matching result outputting operation for outputting a matching result in a format recognizable by the patient-side operator. In order to perform such operations, as shown in FIG. 5, an information acquisition unit 221 and an output control unit 222 are realized as the logical function blocks realized in the calculation apparatus 22. The information acquisition unit 221 acquires from the matching server 10, control information for controlling various operations of the patient-side terminal 20. The output control unit 222 controls the output apparatus 23 to output the matching result in a recognizable form by the patient-side operator based on the control information acquired by the information acquisition unit 221. The information acquisition unit 221 acquires from the input apparatus 24, control information for inputting to the patient-side terminal 20, data required in each processing in the matching server 10. Based on the control information acquired by the information acquisition unit 221, the output control unit 222 outputs (i.e., transmits) the information inputted into the patient-side terminal 20 to the matching server 10 by the communication apparatus 25.

The output apparatus 23 is an apparatus that outputs information to the outside of the patient-side terminal 20 so that the patient side operator can recognize. For example, the output apparatus 23 may output some images. In other words, the output apparatus 23 may include a display apparatus (so-called a display) capable of displaying images. In this case, the patient-side operator can recognize various kinds of information from the matching server 10 using the vision. For example, the output apparatus 23 may output audio. In other words, the output apparatus 23 may include an audio apparatus (so-called a speaker) capable of outputting audio. In this case, the patient-side operator can recognize various kinds of information from the matching server 10 using the auditory sense. For example, the output apparatus 23 may output information on a paper surface. In other words, the output apparatus 23 may include a print apparatus (so-called a printer) capable of printing desired information on the paper surface. In this case, the patient-side operator can recognize various kinds of information from the matching server 10 using the vision.

The input apparatus 24 is an apparatus for receiving input of information to the patient terminal 20 from the outside of the patient terminal 20 (e.g., the patient-side operator). The input apparatus 24 may include, for example, a keyboard, a mouse, a microphone, a touch panel display, and the like. The communication apparatus 25 is an apparatus that enables the patient-side terminal 20 to communicate various kinds of information with the matching server 10. That is, the communication apparatus 25 functions as a communication interface to the matching server 10.

### 4. Configuration of Facility-Side Terminal

Subsequently, the configuration of the facility-side terminal 30 will be described with reference to FIG. 6. FIG. 6 is a block diagram showing an example of the configuration of the facility-side terminal 30. As shown in FIG. 6, the facility-side terminal 30 includes a storage apparatus 31, a calculation apparatus 32, an output apparatus 33, an input apparatus 34, and a communication apparatus 35. The storage apparatus 31, calculation apparatus 32, output apparatus 33, input apparatus 34, and communication apparatus 35 may be connected to each other through a data bus 36. Thus, the configuration of the facility-side terminal 30 may be similar to that of the patient-side terminal 20. In this disclosure, the storage apparatus 21, calculation apparatus 22, output apparatus 23, input apparatus 24, and communication apparatus 25 of the patient-side terminal 20 correspond to the storage apparatus 31, calculation apparatus 32, output apparatus 33, input apparatus 34, and communication apparatus 35 of the facility-side terminal 30 respectively. The operation of each apparatus 31-35 may be similar to the operation of each corresponding apparatus 21-25. Accordingly, the information acquisition unit 221 and the output control unit 222 of the calculation apparatus 22 correspond to an information acquisition unit 321 and an output control unit 322 of the calculation apparatus 32. Further, the patient-side operator corresponds to the facility-side operator, and the patient-side data inputting operation corresponds to a facility-side data inputting operation.

### 5. Operations of the Respective Function Blocks Formed on the Matching Server

In the calculation apparatus 12 of the matching server 10, as described above, the matching processing unit 121, the data learning unit 122, the data item choosing unit 123, the patient-side data input unit 124, the facility-side data input unit 125, and the booking unit 126 are formed as logical functional blocks. The operation of each section 121 to 126 will be described.

The patient-side data input unit 124 generates and outputs the patient matching data D1 to the matching processing unit 121. The patient attribute items Di12 included in the patient matching data D1 are formed by data items, after data item learning processing which will be described below is executed, the data items being chosen based on the learning result. In order to generate the patient matching data D1, the patient-side data input unit 124 acquires from the database 40, information corresponding to the patient attribute-items Di12 included in the patient matching data D1. Out of the information corresponding to the patient attribute items Di12, information available (impossible to be acquired) from the database 40 (for example, information that is not included in in the database 40) is acquired by the patient-side data inputting operation at the patient-side terminal 20. For example, the patient-side data input unit 124 may request the patient-side terminal 20 to input information unavailable (impossible to be acquired) from the database 40 out of the information corresponding to the patient attribute items Di12. Then, the patient-side data input unit 124, by the patient-side data inputting operation performed by the patient-side terminal 20 in response to this input request, may acquire the information impossible to be acquired. Hereinafter, the information corresponding to the patient attribute item(s) Di12 acquired by the patient-side data inputting operation is referred to as “patient supplemental information”. The timing of inputting the patient supplemental information may be appropriate timing prior to the start of the matching processing, or may be appropriate timing after the start of the matching processing. The patient supplemental information inputted to the patient-side terminal 20, may be stored in the storage apparatus 21, for example.

The facility-side data input unit 125 generates the facility matching data D2 corresponding to each of the plurality of medical facilities and outputs the generated facility matching data D2 to the matching processing unit 121. The facility attribute items Di22 included in the facility matching data D2 are formed by data items, after data item learning processing which will be described below is executed, the data items being chosen based on the learning result. In order to generate the facility matching data D2, the facility-side data input unit 125 acquires from the database 40, information corresponding to the facility attribute items Di22 included in the facility matching data D2. Out of the information corresponding to the facility attribute items Di22, information impossible to be acquired from the database 40 (for example, information that is not included in the database 40) is acquired by the facility-side data inputting operation of the facility-side terminal 30. For example, the facility-side data input unit 125 may request the facility-side terminal 30 to input information impossible to be acquired from the database 40 out of information corresponding to the facility attribute items Di22. Then, the facility-side data input unit 125, by the facility-side data input operation performed by the facility-side terminal 30 in response to the input request, may acquire the information impossible to be acquired. Hereinafter, the information corresponding to the facility attribute-item(s) Di22 acquired by the facility-side data inputting operation is referred to as “facility supplemental information”. The timing of inputting the facility supplemental information may be appropriate timing prior to the start of the matching processing, or may be appropriate timing after the start of the matching processing. The facility supplemental information inputted to the facility-side terminal 30, may be stored in the storage apparatus 31, for example.

The matching processing unit 121 performs the matching processing between the patients and the medical facilities. In the matching processing, acquired are the patient matching data D1 from the patient-side data input unit 124 and the facility matching data D2 corresponding to each of the plurality of medical facilities from the facility-side data input unit 125; and at least one recommended facility is selected for the patient by using: the patient item information D12 of the patient matching data D1 as the patient-side matching element; and the facility item information D22 of the facility matching data D2 as the facility-side matching element. When performing the matching processing for each of the plurality of patients, the matching processing unit 121 may give to the matching processing corresponding to each patient, a matching ID that is the identification information for identifying each matching. The patient ID may be utilized as the matching ID. For example, the matching processing unit 121 may store in the storage apparatus 11 in association with the matching ID, the patient attribute items Di12 and facility attribute items Di22 used in the matching.

As the matching method performed by the matching processing unit 121, for example, a rule-based matching method may be applied. When the matching is performed on the rule-based matching, for example, the priority is set in advance with respect to a plurality of criteria based on data items included in the facility attribute items Di22, and the matching is performed by a matching model that is set so that the matching result is obtained in consideration of the priority. As an example of the plurality of criteria, there are for example: a criterion that a medical facility, which has a relatively high past results of the same type of medical examination as the recommended examination, is more likely to be selected as the recommended facility than a medical facility, which has a relatively low past results of the same type of medical examination as the recommended examination; and a criterion that a medical facility, which has a relatively low number of bookings of the same type of medical examination as the recommended examination (i.e., the number of booked medical examinations), is more likely to be selected as the recommended facility than a medical facility, which has a relatively high number of bookings of the same type of medical examination as the recommended examination. One of these criteria may be applied or more than one may be applied.

Specifically, the recommended facility that is the matching result may be selected, for example, by the following procedure. First, for each medical facility, a total of weights (hereinafter referred to as “the total points”) is calculated, the weight being set for each of a plurality of evaluation items (for example, items relating to each criterion described), and a priority order of medical facilities may be set based on the calculated total points. In the setting of the priority order, with respect to medical facilities whose total points are equal to each other, or have a little different from each other (e.g., the difference is less than a predetermined threshold), more than one kind of priority order may be set by changing the priority order. For example, in a case that the total points of medical facility A, the total points of medical facility B, and the total points of medical facility C are 100, 98, and 70 respectively (that is, in a case that the difference in the total points between the medical facility A and the medical facility B is small), two kinds of priority order may be set: a first priority order set in the order of the medical facility A, the medical facility B, and the medical facility C; and a second priority order set in the order of the medical facility B, the medical facility A, and the medical facility C. Then, the matching may be performed for each kind of priority order, and from more than one matching results, the medical facility having the largest total points may be selected to be set as the recommended facility. The weights of the priority order for the static items may be set larger than those for the dynamic items and the wish items, as described above.

As a matching method, for example, matching by a machine learning model that learns on the user base or the contents base may be applied. Also in the matching by the machine learning model, for example, the priority based on the total points described may be set for each of the medical facilities. For learning about matching of the matching processing (hereinafter, referred to as “the matching learning”), the matching processing unit 121 may obtain the patient-side approval result from the patient-side terminal 20 and obtain the facility-side approval result from the facility-side terminal 30. The machine learning model for the matching learning may be provided, for example, so that, by means of the learning, (i) the number of patient-side approval results each indicating approval of the patient side to the matching result increases, (ii) the ratio of the number of patient-side approval results each indicting approval of the patient side to the matching result to the total number of patient-side approval results is greater than a predetermined first threshold value, (iii) the number of patient-side approval results each indicating that disapproval of the patient side to the matching result decreases, (iv) the ratio of the number of patient-side approval results each indicting disapproval of the patient side to the matching result to the total number of patient-side approval results is smaller than a predetermined second threshold value (the second threshold value is equal to or more than the first threshold value), (v) the number of facility-side approval results each indicating approval of the facility side to the matching result increases, (vi) the ratio of the number of facility-side approval results each indicting approval of the facility side to the matching result to the total number of facility-side approval results is greater than a predetermined third threshold value, (vii) the number of facility-side approval results each indicating that disapproval of the facility side to the matching result decreases, and/or (viii) the ratio of the number of facility-side approval results each indicting disapproval of the facility side to the matching result to the total number of facility-side approval results is smaller than a predetermined fourth threshold value (the fourth threshold value is equal to or more than the third threshold value). That is, the machine learning model for the matching learning may be provided to learn so that, for example, at least one of the above-mentioned (i) to (viii) is satisfied by the learning.

The matching processing unit 121 may perform an approval processing to obtain an approval result of the patient with respect to a matching result that is the result of the matching processing (i.e. “the patient-side approval result”) and an approval result of the recommended facility with respect to the matching result (i.e. “the facility-side approval result”). As the approval processing, the matching processing unit 121 may first notify of the matching result, each of the patient-side terminal 20 corresponding to the matched patient and the facility-side terminal 30 corresponding to the matched recommended facility. For example, if there are two or more recommended facilities, the matching result may be notified to the facility-side terminal 30 corresponding to each of the recommended facilities. For example, the patient-side terminal 20 may be notified of the patient-side matching result, and the facility-side terminal 30 may be notified of the facility-side matching result indicating information different from information indicated by the patient-side matching result. Notifying the patient-side terminal 20 of the patient-side matching result may be, that is, notifying the patient of the patient-side matching result. Further, notifying the facility-side terminal 30 of the facility-side matching result may be, that is, notifying the recommended facility of the facility-side matching result. The patient-side matching result may, for example, indicate information about at least one recommended facility selected for the corresponding patient. The information about the recommended facility may include, for example, the facility name and location of the corresponding recommended facility, as well as the facility information corresponding to the respective wish items in the patient attribute items Di12. The facility-side matching result may indicate, for example, information about the patient for whom the corresponding facility has been selected. The information about the patient may include, for example, the name, address, and age of the corresponding patient, as well as patient information corresponding to the respective wish items in the facility attribute items Di22. In addition, the information indicated by the facility-side matching result may be also notified to the patient-side terminal 20. Further, the information indicated by the patient-side matching result may be also notified to the facility-side terminal 30.

The matching processing unit 121 requests the patient-side terminal 20 to set a patient-side response to the patient-side matching result and requests the facility-side terminal 30 to set a facility-side response to the facility-side matching result. The patient-side response indicates approval or disapproval to the patient-side matching result on the patient side. The facility-side response indicates approval or disapproval to the facility-side matching result on the facility side. For example, the matching processing unit 121 causes the patient-side terminal 20 to perform the matching result outputting operation (i.e., to present the patient-side matching result to the patient-side operator) and causes the patient-side operator to input the response (the approval or the disapproval) to the patient-side matching result to set the patient-side response. Similarly, for example, the matching processing unit 121 causes the facility-side terminal 30 to perform the matching result outputting operation and causes the facility-side operator to input the response (the approval or the disapproval) to the facility-side matching result to set the facility-side response.

The booking unit 126 automatically making a booking in a case that the both responses of the patient and the recommended facility are “approval.” Thus, the booking unit 126 automatically makes a booking for the patient to the medical facility approved by the patient. The booking unit 126 performs processing, for example, as follows. The booking unit 126 acquires the patient-side response set at the patient-side terminal 20 that has received the patient-side matching result. If two or more recommended facilities are selected and presented for the patient, in the patient-side response, the recommended facilities approved by the patient (e.g., in order of preference) may be indicated, for example. The booking unit 126 acquires the facility-side response set at the facility-side terminal 30 that has received the facility-side matching result.

If both of the patient-side and facility-side responses indicate “approval”, the booking unit 126 performs the booking processing with respect to the corresponding matching result. For example, the booking date and time are determined based on the “schedule” of the patient attribute items Di12 and the “operational status” of the facility attribute items Di22. After determining the booking date and time, for example, a booking result indicating the booking content is transmitted to each of: the patient-side terminal 20 corresponding to the patient who is the booking target; and the facility-side terminal 30 corresponding to the recommended facility which is the booking target. For example, the patient-side booking result may be transmitted to the patient-side terminal 20, and the facility-side booking result may be transmitted to the facility-side terminal. In the patient-side booking result, there may be indicated, for example, the facility name, the location, the type of medical examination, the booking date and time, and the like with respect to the recommended facility which is the booking target. In the facility-side booking result, there may be indicated, for example, the name, age, the address, the type of medical examination, the booking date and time, and the like with respect to the patient who is the booking target. The information included in the facility-side booking result may be also transmitted to the patient-side terminal 20 as the booking result. The information included in the patient-side booking result may be also transmitted to the facility-side terminal 30 as the booking result.

The data learning unit 122 learns about the patient attribute items and facility attribute items necessary or useful for matching so that the matching result is likely to be approved, based on the patient-side approval results and facility-side approval results to the matching results. The data learning unit 122 performs processing as follows, for example.

The data learning section 122 acquires the patient-side approval result from the patient-side terminal 20 and acquires the facility-side approval result from the facility-side terminal 30. The patient-side approval result includes, for example, the matching ID and the patient-side response. The facility-side approval result includes, for example, the matching ID and the facility-side response. The data learning unit 122 performs data item learning processing based on the acquired patient-side approval result and the acquired facility-side approval result to learn about the patient attribute items and facility attribute items necessary or useful for matching. For example, the data learning unit 122 learns about the patient attribute items determined to be necessary for matching or to have a relatively high probability of helping in matching (i.e., have a relatively high necessary level for matching) based on the patient-side approval result and the patient attribute items Di12 used for the matching. Further, the data learning unit 122, for example, learns about the facility attribute items determined to be necessary for matching or to have a relatively high probability of helping in matching (i.e., have a relatively high necessary level for matching) based on the facility-side approval result and the facility attribute items Di22 used for the matching.

For example, in a case that the probability of obtaining approval to a matching result caused by one data item within the plurality of patient attribute items Di12 (i.e., a recommended facility matched by using the one data item) exceeds a predetermined criterion, the one data item may be determined as the patient attribute item “that is necessary for matching or has a relatively high probability of helping in matching” (i.e., that has a relatively high necessary level). For example, in a case that the probability of obtaining approval to a matching result caused by one data item within the plurality of facility attribute items Di22 (i.e., a recommended facility matched by using the one data item) exceeds a predetermined criterion, the one data item may be determined as the facility attribute item “that is necessary for matching or has a relatively high probability of helping in matching” (i.e., that has a relatively high necessary level). The data items that are necessary for matching or have a relatively high probability of helping in matching, may include one data item satisfying a predetermined criterion for item choice. The predetermined criterion for item choice, for example, may include a first criterion that the ratio of the number of patient-side approval results indicating that the patient side have approved the matching result based on the one data item, to a total number of patient-side approval results is greater than a predetermined fifth threshold. The predetermined criterion for item choice, for example, may include a second criterion that the ratio of the number of patient-side approval results indicating that the patient side have disapproved the matching result based on the one data item, to a total number of patient-side approval results is smaller than a predetermined sixth threshold (the sixth threshold is equal to or less than the fifth threshold). The predetermined criterion for item choice, for example, may include a third criterion that the ratio of the number of facility-side approval results indicating that the facility side have approved the matching result based on the one data item, to a total number of facility-side approval results is greater than a predetermined seventh threshold. The predetermined criterion for item choice, for example, may include a fourth criterion that the ratio of the number of facility-side approval results indicating that the facility side have disapproved the matching result based on the one data item, to a total number of facility-side approval results is less than a predetermined eighth threshold (the eighth threshold is equal to or less than the seventh threshold). The fifth to eighth thresholds correspond to the above-described first to fourth thresholds respectively. The fifth to eighth thresholds may be, for example, the same value as the first to fourth thresholds respectively.

The data learning unit 122 may include, for example, a rule-based calculation model that outputs the necessary level of each data item used in the matching when the patient-side approval result and the facility-side approval result are input. In this case, the data learning unit 122 may perform learning processing based on the patient-side approval result and the facility-side approval result, to update the rule indicating operation details of the rule-based calculation model so that the above-described predetermined criterion for item choice is satisfied. The calculation model may be a machine learning model capable of machine learning (e.g., a machine learning model using a neural network). In this case, the data learning unit 122 may perform learning processing using the patient-side approval result and the facility-side approval result, to set parameters (for example, at least one of the weights and biases of the neural network) of the machine learning model so that the above-described predetermined criterion for item choice is satisfied (for example, so that the loss-function based on the criterion for item choice is minimized). Alternatively, in addition to or in place of the aforementioned learning processing using the criterion for item choice, the data learning unit 122 may set the parameters of the machine learning model such that the output of the machine learning model (i.e., the predicted value obtained from the machine learning model) is close to the correct answer value, for example, by using the learning data where: the patient attribute items Di12 used for matching corresponding to a case that the patient-side response indicates “approval” are regarded as correct answer data; and the patient attribute items Di12 used for matching corresponding to a case that the patient-side response indicates “disapproval” are regarded as incorrect answer data. Further, the data learning unit 122 may set the parameters of the machine learning model such that the output of the machine learning model (i.e., the predicted value obtained from the machine learning model) is close to the correct answer value, by using the learning data where: the facility attribute items Di22 used for matching corresponding to a case that the facility-side response indicates “approval” are regarded as correct answer data; and the facility attribute items Di22 used for matching corresponding to a case that the facility-side response indicates “disapproval” are regarded as incorrect answer data.

By the learning processing of the data learning unit 122 as described above, the patient attribute items Di12 that would cause the matching result that is likely to be approved by the patient; and the facility attribute items Di22 that would cause the matching result that is likely to be approved by the medical facility are learned as the patient attribute items Di12 and facility attribute items Di22 necessary for matching,

The data learning unit 122 may perform the learning processing based on the patient-side approval result and the facility-side approval result that satisfy a predetermined learning timing condition. For example, the learning timing condition may be “each matching processing” as the first learning timing. In this case, the data learning unit 122 may perform the learning processing every time the patient-side approval result and the facility-side approval result are acquired. The learning timing condition may be “the number of patient-side approval results and facility-side approval results” as the second learning timing. In this case, the data learning unit 122 may perform the learning processing after acquiring a predetermined number of patient-side approval results and facility-side approval results. When the second learning timing is set as the learning timing condition, the data learning unit 122 may, for example, store in the storage apparatus 11, more than one patient-side approval result and more than one facility-side approval result until the number of them reaches the predetermined number.

In addition, when the learning timing condition is “every predetermined time” as the third learning timing, the data learning unit 122 may perform the learning processing based on the patient-side approval results and facility-side approval results acquired within the predetermined time. When the third learning timing is set as the learning timing condition, the data learning unit 122 may, for example, store in the storage apparatus 11, the patient-side approval results and the facility-side approval results generated within the predetermined time. When the data item learning is performed based on a plurality of patient-side approval results and a plurality of facility-side approval results, the learning results with high accuracy can be efficiently obtained without a case that the learning result is affected by one special case, for example.

The data learning unit 122 may perform the data item learning for each type of information (hereinafter, referred to as “the information type”) corresponding to the coincidence-required item out of the patient attribute items Di12 and the facility attribute items Di22, the coincidence-required item requiring coincidence in the matching processing. For example, when the coincidence-required item is the medical examination type (that is, “the patient med-exam type” and “the facility med-exam type”), the data learning section 122 may learn the patient attribute items and facility attribute items necessary for matching for each information type of the medical examination type. For example, the data learning unit 122 may store in the storage apparatus 11, the necessary level of each data item obtained as the learning result, in association with the information type of the coincidence-required item (for example, the medical examination type). In addition, the data learning unit 122 may perform the data item learning for each information type of any one of plurality of patient-attribute items Di12 (hereinafter, referred to as “the learning reference item”). For example, when the learning reference item is “age”, the data learning unit 122 may perform the data item learning for each information type of “age” (for example, low age, middle age, high age, etc.). For example, the data learning unit 122 may store in the storage apparatus 11, the necessary level of each data item obtained as the learning result, in association with the information type of the learning reference item. With respect to a data item that is always necessary for the matching processing (for example, the coincidence-required item), the data learning unit 122 is not required to set the data item to the target of the data item learning processing.

The data item choosing unit 123 chooses a plurality of patient attribute items Di12 and a plurality of facility attribute items Di22 to be used in the matching processing based on the learning result of the data item learning processing described above. The data item choosing unit 123 may choose, for example, from the patient attribute item list (FIG. 2B), some data items as the patient attribute items Di12 to be used in the matching processing, based on the learning result of the data item learning processing. In addition, the data item choosing unit 123 may choose, for example, from the facility attribute item list (FIG. 3B), some data items as the facility attribute items Di22 to be used in the matching processing, based on the learning result of the data item learning processing. In a case that obtained as the learning result of the data item learning processing is, for example, the necessary level for matching of each data item, the data item choosing unit 123 may choose the data items each having the necessary level that is equal to or more than a predetermined threshold from the data items in the patient attribute item list and the data items in the facility attribute item list. The choosing may be performed based on the necessary level of each data item, the necessary level depending on the information type of information corresponding to the coincidence-required item. The choosing may be performed based on the necessary level of each data item, the necessary level depending on the information type of information corresponding to the learning reference item. The patient-side threshold which is the threshold of necessary level with respect to the patient attribute items Di12 and the facility-side threshold which is the threshold of necessary level with respect to the facility attribute items Di22, may be the same as or different from each other. Further, at least one of the patient-side threshold and the facility-side threshold may vary depending on, for example, the number of executions of the data item learning processing. The data item choosing unit 123 may always choose a data item that is the coincidence-required item. The data item choosing unit 123 may always choose a data item that is always required in the matching processing like the coincidence-required item. The necessary level of a data item which should be always chosen may be set to, for example, the maximum value by the data learning unit 122.

As described above, in the data item choosing unit 123, data items as the patient attribute items Di12 and data items as the facility attribute items Di22, both of which are used in the matching processing by the matching processing unit 121, are chosen. The choosing by the data item choosing unit 123 is performed based on the learning result of the data item learning processing performed by the data learning unit 122. Therefore, by repeating the data item learning processing, it is possible to increase the choosing accuracy of data items to be chosen as the patient-attribute items Di12 by the data item choosing unit 123, and also increase the choosing accuracy of data items to be chosen as the facility-attribute items Di22.

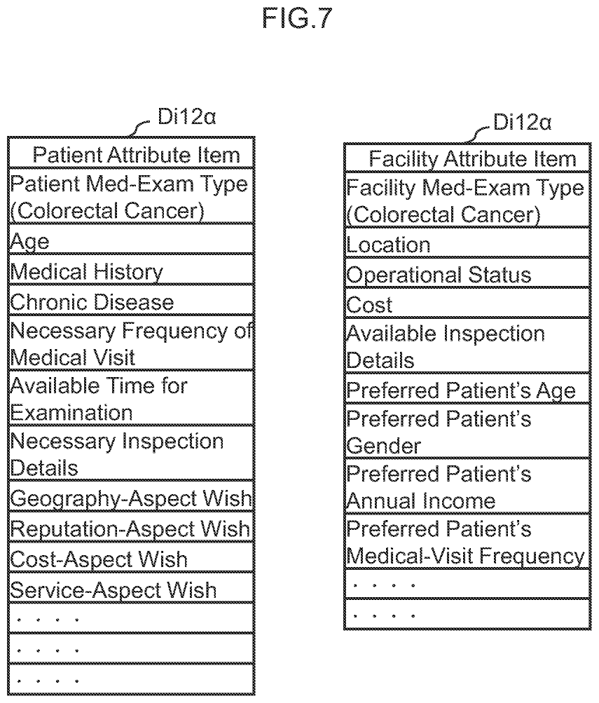

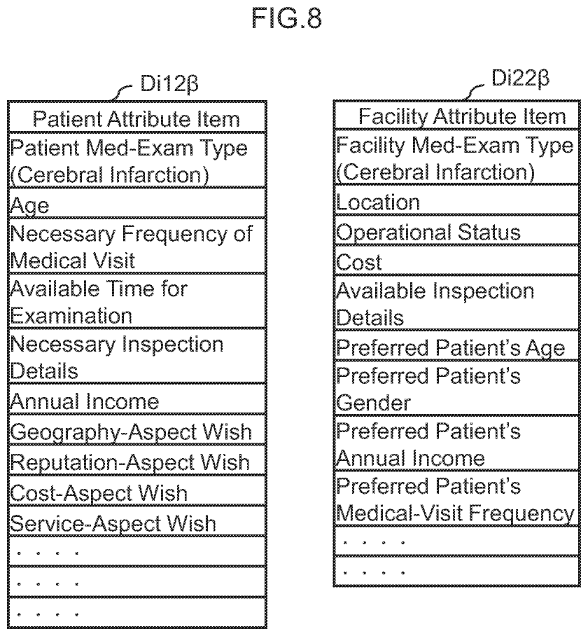

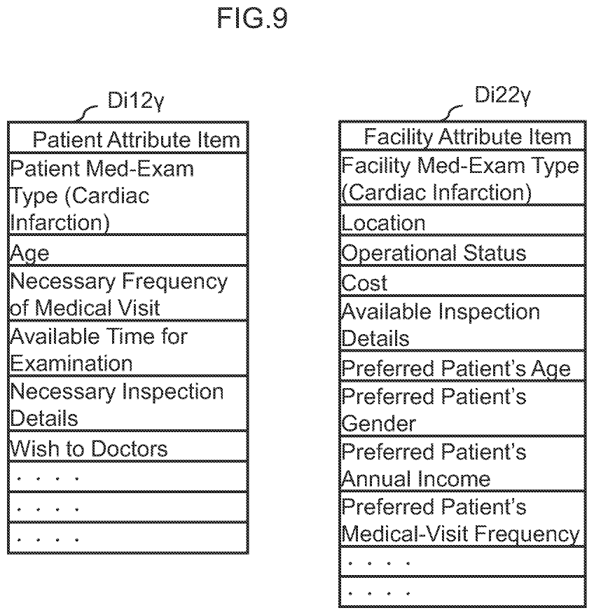

FIG. 7 to FIG. 9 show examples of the patient attribute items Di12 and facility attribute items Di22 chosen by the data item choosing unit 123 based on the learning result of the data item learning processing, which has been executed with respect to the patient attribute items Di12 and the facility attribute items Di22 for each information type of the medical examination type as the coincidence-required item (i.e., “the patient med-exam type” and “the facility med-exam type”). FIG. 7 shows an example of the patient attribute items Di12α and facility attribute items Di22α that are the patient attribute items Di12 and facility attribute items Di22 chosen with respect to the medical examination for bowel cancer in a case that the medical examination type is the medical examination for bowel cancer. FIG. 8 is an example of the patient attribute items Di12β and facility attribute items Di22β that are the patient attribute items Di12 and facility attribute items Di22 chosen with respect to the medical examination for cerebral infarction in a case that the medical examination type is the medical examination for cerebral infarction. FIG. 9 shows an example of the patient attribute items Di12γ and facility attribute items Di22γ that are the patient attribute items Di12 and facility attribute items Di22 chosen with respect to the medical examination for cardiac infarction in a case that the medical examination type is the medical examination for cardiac infarction.

### 6. Processing Flow in Matching System

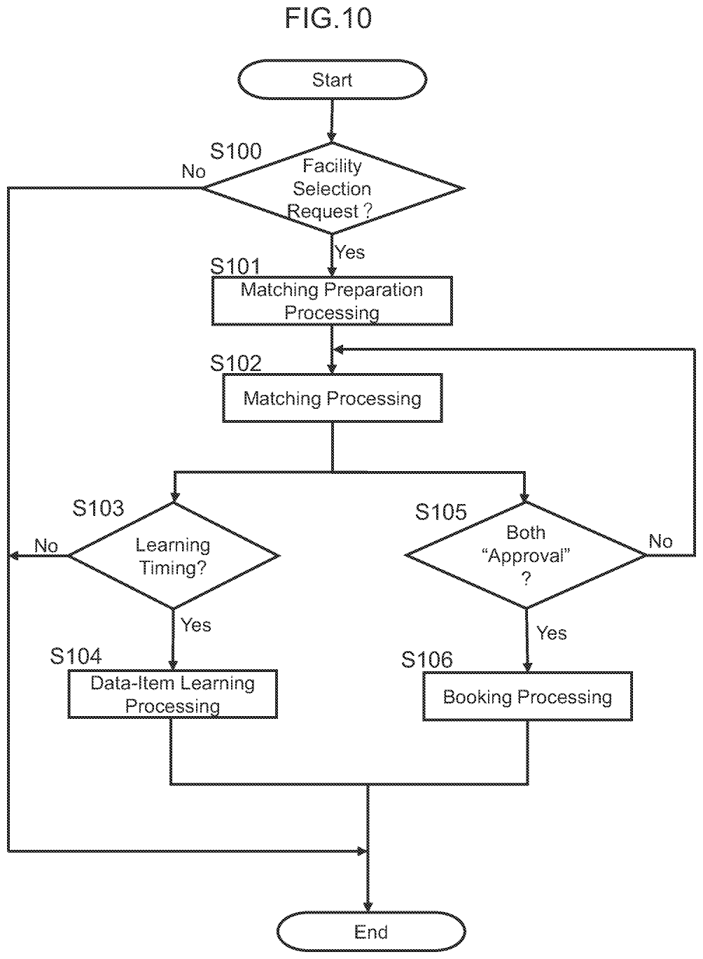

An exemplary flow of processes in the matching system 1 will be described with reference to FIG. 10. Here, the case that the “patient med-exam type” and the “facility med-exam type” are the coincidence-required item described above will be described. The processes shown in FIG. 10 are executed by the calculation apparatus 12 of the matching server 10. First, the matching server 10 determines whether or not a facility selection request (i.e., a matching request) has been received from the patient-side terminal 20 (step S100). The facility selection requirement includes, for example, the patient basic information D11 and the recommended examination with respect to the patient who is the matching target. If it is determined that the facility selection request has not been received (step S100: No), the matching server 10 terminates the current processing routine. If it is determined that the facility selection request has been received (step S100: Yes), the matching server 10 performs a matching preparation processing (step S101).

In the matching preparation processing, data item choosing processing and input data processing are included. In the data item choosing processing, the data items constituting the patient attribute items Di12 and the data items constituting the facility attribute items Di22 to be used in the matching processing are chosen based on the learning results of the data item learning processing by the data item choosing unit 123. Therefore, the patient attribute items Di12 included in the patient matching data D1 are the data items chosen in the data item choosing processing. Also, the facility attribute items Di22 included in the facility matching data D2 are the data items chosen in the data item choosing processing. For example, in a case that the recommended examination included in the facility selection requirement is the “medical examination for colorectal cancer”, the patient matching data D1 includes the patient attribute items Di12α (FIG. 7) chosen, and the facility matching data D2 includes the facility attribute items Di22α (FIG. 7) chosen. The data item choosing unit 123 may, for example, acquire information corresponding to the learning reference item from the facility selection request and perform the data item choosing processing based on the necessary level depending on the information corresponding to the learning reference item acquired. In the input data processing, the patient matching data D1 is generated by the patient-side data input unit 124, and the facility matching data D2 corresponding to each of the plurality of medical facilities is generated by the facility-side data input unit 125. In a case that the med-exam type is set as the coincidence-required item, there may be generated the facility matching data D2 where the recommended examination included in the facility selection request is coincident with the facility med-exam type of the facility attribute item Di22.

In generating the patient matching data D1, the patient side data input unit 124 acquires from the patient-side terminal 20, the patient supplemental information that cannot be acquired from the database 40. When generating the facility matching data D2, the facility-side data input unit 125 acquires from the facility-side terminal 30, the facility supplemental information that cannot be acquired from the database 40.

After the patient matching data D1 and the facility matching data D2 are generated, the matching server 10 performs the matching processing described above by the matching processing unit 121 (step S102). The matching processing unit 121 sends the matching result (i.e., the patient-side matching result) to the patient-side terminal 20 to make the patient-side terminal 20 set a patient-side response (approval or disapproval) to the matching result. Further, the matching processing unit 121, as described above, sends the matching result (i.e., the facility-side matching result) to the facility-side terminal 30 to make the facility-side terminal 30 set a facility-side response (approval or disapproval) to the matching result.

After the matching processing, the data learning unit 122 acquires the patient-side approval result from the patient-side terminal 20 and acquires the facility-side approval result from the facility-side terminal 30 to determine whether or not the learning timing condition described above is satisfied (step S103). When it is determined that the learning timing condition is not satisfied (step S103: No), the data learning section 122, without performing the learning processing, stores in, for example, the storage apparatus 11 the acquired patient-side approval result and the acquired facility-side approval result, and then terminates the processing routine. When it is determined that the learning timing condition is satisfied (Step S103: Yes), the data learning unit 122 performs the data item learning processing described above, based on the patient-side approval result and facility-side approval result (for example, the patient-side approval result and facility-side approval result which have been acquired, and the patient-side approval result and facility-side approval result stored in the storage apparatus 11), which satisfies the learning timing condition (Step S104). The matching server 10 terminates the processing routine after the data-item learning processing ends.

In addition, after the matching processing, the booking unit 126 acquires the patient-side response from the patient-side terminal 20 and acquires the facility-side response from the facility-side terminal 30 to determine whether both the patient-side response and the facility-side response indicate “approval” (step S105). When both of the responses indicate “approval” (step S105: Yes), the booking unit 126 performs the booking processing described above (step S106). The matching server 10 terminates the processing routine after executing the booking processing. When at least one of the patient-side response and the facility-side response indicates “disapproval” (step S105: No), the matching server 10 may, for example, perform the matching processing again by the matching processing unit 121.

For example, the patient attribute item list may include as its data item in place of/in addition to the “patient med-exam type”, a “clinical department” where the patient plans to go, and the facility attribute item list may include as its data item in place of/in addition to the “facility med-exam type”, a “clinical department” that the medical facility provides. In this case, the data learning unit 122 may set the coincidence-required item to “the clinical department” to learn for each clinical department, the patient attribute items and facility attribute items necessary for the matching processing. In a case that the patient-side terminal 20 and the facility-side terminal 30 are physically one terminal, a data input unit where the patient-side data input unit 124 and the facility-side data input unit 125 are combined may be formed as a logical function block in the calculation apparatus 12 of the matching server 10. The patient-side terminal 20 where the patient-side data inputting operation is performed and the patient-side terminal 20 where the patient-side matching result is notified, may be physically different from each other. The facility-side terminal 30 where the facility-side data inputting operation is performed and the facility-side terminal 30 where the facility-side matching result is notified, may be physically different from each other.

### SUPPLEMENTARY NOTE

With respect to the example embodiments described above, they may be further described as in supplementary notes below.

### Supplementary Note 1

A medical facility matching system described in Supplementary Note 1 is a medical facility matching system comprising: a matching processing unit that is configured to perform matching processing to select as a matching result, at least one medical facility to be recommended for a patient from a plurality of medical facilities, based on patient item information corresponding to a plurality of patient attribute items with respect to the patient and facility item information corresponding to a plurality of facility attribute items with respect to each of the plurality of medical facilities; a data learning unit that is configured to learn about the plurality of patient attribute items necessary for the matching processing so that the matching result is likely to be approved by the patient, and learn about the plurality of facility attribute items necessary for the matching processing so that the matching result is likely to be approved by the medical facility selected; and a data item choosing unit that is configured to choose based on a learning result of the data learning unit, the plurality of patient attribute items to be used in the matching processing and the plurality of facility attribute items to be used in the matching processing, wherein the matching processing unit is configured to perform the matching processing based on the patient item information corresponding to the plurality of patient attribute items chosen and the facility item information corresponding to the plurality of facility attribute items chosen.

According to the medical facility matching system described in Supplementary Note 1, the learning about the plurality of patient attribute items and plurality of facility attribute items necessary for the matching processing is executed so that the approval to the matching result is likely to be obtained, based on the previous matching results. The patient attribute items and facility attribute items chosen based on the learning result will be used in future matching processing. Due to this, it is possible to weed out unnecessary patient attribute items and facility attribute items from the matching elements which are referenced in the matching processing, and thereby make the matching processing more efficient.

### Supplementary Note 2

The matching system described in Supplementary Note 2 is the medical facility matching system according to Supplementary Note 1, wherein the data learning unit is configured to learn about the plurality of facility attribute items necessary for the matching processing and the plurality of patient attribute items necessary for the matching processing, based on whether or not the matching result has been approved by each of the patient and the at least one medical facility selected.

According to the medical facility matching system described in Supplementary Note 2, the learning by the data learning unit can be executed based on whether or not each of the selected patient and selected medical facility as the matching result has approved the matching result. The data learning unit may learn about the plurality of facility attribute items and plurality of patient attribute items necessary for the matching processing, based on at least one of the matching results satisfying a predetermined learning timing condition. If the learning timing condition is set to a desired timing, it is possible to make the data learning unit learn at the desired learning timing based on the matching result satisfying the learning timing condition.

### Supplementary Note 3

The matching system described in Supplementary Note 3 is the medical facility matching system according to Supplementary Note 1 or 2, wherein the plurality of facility attribute items include at least one item with respect to a wish of the medical facility to the patient, and the plurality of patient attribute items include at least one item with respect to a wish of the patient to the medical facility.

According to the medical facility matching system described in Supplementary Note 3, with respect to the patient and the facility each, it is possible to use as the matching element(s), not only objective information but also information on the desire to a partner to be selected.

### Supplementary Note 4

The matching system described in Supplementary Note 4 is the medical facility matching system according to any one of Supplementary Notes 1 to 3, wherein the data learning unit is configured to learn about the plurality of patient attribute items necessary for the matching processing and the plurality of facility attribute items necessary for the matching processing, for each type of information corresponding to any one of the plurality of patient attribute items.

According to the medical facility matching system described in Supplementary Note 4, the learning by the data learning unit is executed for each type of information corresponding to the patient attribute item. Thereby, it is possible to obtain depending on the type, the learning results with respect to the patient attribute items and facility attribute items necessary for the matching processing.

### Supplementary Note 5

The matching system described in Supplementary Note 5 is the medical facility matching system according to any one of Supplementary Notes 1 to 4, wherein the data learning unit is configured to learn about the plurality of patient attribute items necessary for the matching processing and the plurality of facility attribute items necessary for the matching processing, for each type of information corresponding to a coincidence-required item, the coincidence-required item requiring that information corresponding to the facility attribute item and information corresponding to the patient attribute item are coincident with each other in the matching processing.

According to the medical facility matching system described in Supplementary Note 5, the learning by the data learning unit is executed for each type of information corresponding to the coincidence-required item. As a result, it is possible to obtain depending on the type, the learning results with respect to the patient attribute items and facility attribute items necessary for the matching processing.

### Supplementary Note 6

The matching system described in Supplementary Note 6 is the matching system according to any one of Supplementary Notes 1 to 5, wherein the plurality of patient attribute items include a patient med-exam type indicating a medical examination type which the patient plans to undergo and the plurality of facility attribute items include a facility med-exam type indicating a medical examination type which is available in the medical facility, and the data learning unit is configured to learn about the plurality of facility attribute items necessary for the matching processing and the plurality of patient attribute items necessary for the matching processing, for each medical examination type.

According to the medical facility matching system described in Supplementary Note 6, it is possible to learn about the patient attribute items and facility attribute items necessary for matching processing by narrowing down the matching processing to each med-exam type, and thereby efficiently performing the learning processing. The data learning unit may learn about the patient attribute items and facility attribute items, based on the coincidence-required item within the patient attribute items and the facility attribute items, which requires the coincidence in the matching processing. As the coincidence-required items, for example, the medical examination type and the clinical department could be considered.

### Supplementary Note 7

The medical facility matching system described in Supplementary Note 7 is the medical facility matching system according to any one of Supplementary Notes 1 to 6, wherein the matching processing unit is configured to perform the matching processing utilizing a machine learning model, the machine learning model having been learned about matching in the matching processing based on whether or not the matching result has been approved by each of the patient and the at least one medical facility selected.

According to the medical facility matching system described in Supplementary Note 7, a machine learning model is learned regarding matching of the matching processing, based on the approval result to the matching result of each of the patient and the selected medical facility. Then, the matching processing unit can perform the matching processing using the machine learning model.

### Supplementary Note 8

The medical facility matching system described in Supplementary Note 8 is the medical facility matching system according to any one of Supplementary Notes 1 to 7, further comprising: a database that includes patient relating information relating to the patient and facility relating information relating to each of the plurality of medical facilities; and a data input unit that is configured to acquire the patient item information and facility item information to be used in the matching processing, from the patient relating information and facility relating information in the database.

According to the medical facility matching system described in Supplementary Note 8, the patient item information can be obtained from the database including the patient relating information to be formed, and also, the facility item information can be obtained from the database including the facility relating information to be formed. Thereby, a database dedicated to the patient item information and/or a database dedicated to the facility item information are/is not required, and it is possible to enable the use of a wide range of databases, for example, databases managed by other systems.

### Supplementary Note 9

The medical facility matching system described in Supplementary Note 9 is the medical facility matching system according to Supplementary Note 8, including: a facility-side terminal capable of accepting operations relating to the medical facility; a patient-side terminal capable of accepting operations relating to the patient; and a server capable of data communication with the facility-side terminal and the patient-side terminal, wherein the matching processing unit, the data learning unit, and the data input unit are provided by the server, the data input unit comprises: a facility-side data input unit that is configured to acquire the facility item information from the facility relating information in the database; and a patient-side data input unit that is configured to acquire the patient item information from the patient relating information in the database, and wherein the facility-side data input unit is further configured to acquire by input to the facility-side terminal, the facility item information unavailable from the database, and the patient-side data input unit is further configured to acquire by input to the patient-side terminal, the patient item information unavailable from the database.

According to the medical facility matching system described in Supplementary Note 9, the facility-side terminal accepts operations relating to the medical facility, and the patient-side terminal accepts operations relating to the patient. In other words, the operations relating to the medical facility and the operations relating to the patient can be performed at different terminals from each other, and thus, it is possible to efficiently perform various operations which are required on the patient side and the medical facility side, for example.

### Supplementary Note 10

A medical facility matching method described in Supplementary Note 10 is a medical facility matching method of making a computer: perform matching processing to select as a matching result, at least one medical facility to be recommended for a patient from a plurality of medical facilities, based on patient item information corresponding to a plurality of patient attribute items with respect to the patient and facility item information corresponding to a plurality of facility attribute items with respect to each of the plurality of medical facilities; learn about the plurality of patient attribute items necessary for the matching processing so that the matching result is likely to be approved by the patient, and learn about the plurality of facility attribute items necessary for the matching processing so that the matching result is likely to be approved by the medical facility selected; choose based on a learning result with respect to the plurality of patient attribute items and the plurality of facility attribute items, the plurality of patient attribute items to be used in the matching processing and the plurality of facility attribute items to be used in the matching processing; and perform the matching processing based on the patient item information corresponding to the plurality of patient attribute items chosen and the facility item information corresponding to the plurality of facility attribute items chosen.

According to the medical facility matching method described in Supplementary Note 10, as with the medical facility matching system described in the Supplementary Note 1, it is possible to weed out from the matching elements, unnecessary patient attribute items and facility attribute items for the matching processing, and thereby make the matching processing more efficient.

### Supplementary Note 11

A recording medium described in Supplementary Note 11 is a recording medium on which a computer program is recorded, the computer program being configured to allow a computer to function as: a matching processing unit that is configured to perform matching processing to select as a matching result, at least one medical facility to be recommended for a patient from a plurality of medical facilities, based on patient item information corresponding to a plurality of patient attribute items with respect to the patient and facility item information corresponding to a plurality of facility attribute items with respect to each of the plurality of medical facilities; a data learning unit that is configured to learn about the plurality of patient attribute items necessary for the matching processing so that the matching result is likely to be approved by the patient, and learn about the plurality of facility attribute items necessary for the matching processing so that the matching result is likely to be approved by the medical facility selected; and a data item choosing unit that is configured to choose based on a learning result of the data learning unit, the plurality of patient attribute items to be used in the matching processing and the plurality of facility attribute items to be used in the matching processing, wherein the matching processing unit is configured to perform the matching processing based on the patient item information corresponding to the plurality of patient attribute items chosen and the facility item information corresponding to the plurality of facility attribute items chosen.

According to the computer program recorded in the recording medium described in Supplementary Note 11, it is possible to realize the medical facility matching system described in Supplementary Note 1.

The present invention can be changed as appropriate in range which is not contrary to range of the claims and the subject matter of this invention which can be read from the entire specification, and medical facility matching systems, medical facility matching methods, and recording medium with such changes are also included in the technical concepts of the present invention.

### DESCRIPTION OF REFERENCE SIGNS

1 Medical facility matching system
10 Matching server
20 Patient-side terminal
30 Facility-side terminal
40 Database
121 Matching processing unit
122 Data learning unit
123 Data item choosing unit
124 Patient-side data input unit
125 Facility-side data input unit
126 Booking unit

What is claimed is:

1. A medical facility matching system comprising:
at least one memory configured to store instructions; and
at least one processor configured to execute the instructions to:
perform matching processing to select as a matching result, at least one medical facility to be recommended for a patient from a plurality of medical facilities, based on patient item information corresponding to a plurality of patient attribute items with respect to the patient and facility item information corresponding to a plurality of facility attribute items with respect to each of the plurality of medical facilities;
learn, by machine learning, about the plurality of patient attribute items necessary for the matching processing so that the matching result is likely to be approved by the patient, and learn, by machine learning, about the plurality of facility attribute items necessary for the matching processing so that the matching result is likely to be approved by the medical facility selected;
choose based on a learning result with respect to the plurality of patient attribute items and the plurality of facility attribute items, the plurality of patient attribute items to be used in the matching processing and the plurality of facility attribute items to be used in the matching processing; and
perform the matching processing based on the patient item information corresponding to the plurality of patient attribute items chosen and the facility item information corresponding to the plurality of facility attribute items chosen.

2. The medical facility matching system according to claim 1, wherein
the at least one processor is further configured to execute the instructions to:
learn about the plurality of facility attribute items necessary for the matching processing and the plurality of patient attribute items necessary for the matching processing, based on whether or not the matching result has been approved by each of the patient and the at least one medical facility selected.

3. The medical facility matching system according to claim 1, wherein
the plurality of facility attribute items include at least one item with respect to a wish of the medical facility to the patient, and
the plurality of patient attribute items include at least one item with respect to a wish of the patient to the medical facility.

4. The medical facility matching system according to claim 1, wherein
the at least one processor is further configured to execute the instructions to
learn about the plurality of patient attribute items necessary for the matching processing and the plurality of facility attribute items necessary for the matching processing, for each type of information corresponding to any one of the plurality of patient attribute items.

5. The medical facility matching system according to claim 1, wherein
the at least one processor is further configured to execute the instructions to
learn about the plurality of patient attribute items necessary for the matching processing and the plurality of facility attribute items necessary for the matching processing, for each type of information corresponding to a coincidence-required item, the coincidence-required item requiring that information corresponding to the facility attribute item and information corresponding to the patient attribute item are coincident with each other in the matching processing.

6. The medical facility matching system according to claim 1, wherein
the plurality of patient attribute items include a patient med-exam type indicating a medical examination type which the patient plans to undergo and the plurality of facility attribute items include a facility med-exam type indicating a medical examination type which is available in the medical facility, and
the at least one processor is further configured to execute the instruction to
learn about the plurality of facility attribute items necessary for the matching processing and the plurality of patient attribute items necessary for the matching processing, for each medical examination type.

7. The medical facility matching system according to claim 1, wherein
the at least one processor is further configured to execute the instructions to
perform the matching processing utilizing a machine learning model, the machine learning model having been learned about matching in the matching processing based on whether or not the matching result has been approved by each of the patient and the at least one medical facility selected.

8. The medical facility matching system according to claim 1, further comprising:
a database that includes patient relating information relating to the patient and facility relating information relating to each of the plurality of medical facilities; and
the at least one processor is further configured to execute the instructions to acquire the patient item information and facility item information to be used in the matching processing, from the patient relating information and facility relating information in the database.

9. The medical facility matching system according to claim 8, including:
a facility-side terminal capable of accepting operations relating to the medical facility; a patient-side terminal capable of accepting operations relating to the patient; and a server capable of data communication with the facility-side terminal and the patient-side terminal, wherein
the at least one processor is provided by the server, and
the at least one processor is further configured to execute the instructions to
acquire the facility item information from the facility relating information in the database; and
acquire the patient item information from the patient relating information in the database,
and wherein the at least one processor is further configured to execute the instructions to acquire by input to the facility-side terminal, the facility item information unavailable from the database; and acquire by input to the patient-side terminal, the patient item information unavailable from the database.

10. A medical facility matching method of making a computer:
perform matching processing to select as a matching result, at least one medical facility to be recommended for a patient from a plurality of medical facilities, based on patient item information corresponding to a plurality of patient attribute items with respect to the patient and facility item information corresponding to a plurality of facility attribute items with respect to each of the plurality of medical facilities;
learn, by machine leaning, about the plurality of patient attribute items necessary for the matching processing so that the matching result is likely to be approved by the patient, and learn, by machine learning, about the plurality of facility attribute items necessary for the matching processing so that the matching result is likely to be approved by the medical facility selected;
choose based on a learning result with respect to the plurality of patient attribute items and the plurality of facility attribute items, the plurality of patient attribute items to be used in the matching processing and the plurality of facility attribute items to be used in the matching processing; and
perform the matching processing based on the patient item information corresponding to the plurality of patient attribute items chosen and the facility item information corresponding to the plurality of facility attribute items chosen.

11. A non-transitory recording medium on which a computer program is recorded, the computer program being configured to allow a computer to:
perform matching processing to select as a matching result, at least one medical facility to be recommended for a patient from a plurality of medical facilities, based on patient item information corresponding to a plurality of patient attribute items with respect to the patient and facility item information corresponding to a plurality of facility attribute items with respect to each of the plurality of medical facilities;
learn, by machine learning, about the plurality of patient attribute items necessary for the matching processing so that the matching result is likely to be approved by the patient, and learn, by machine learning, about the plurality of facility attribute items necessary for the matching processing so that the matching result is likely to be approved by the medical facility selected;
choose based on a learning result with respect to the plurality of patient attribute items and the plurality of facility attribute items, the plurality of patient attribute items to be used in the matching processing and the plurality of facility attribute items to be used in the matching processing; and
perform the matching processing based on the patient item information corresponding to the plurality of patient attribute items chosen and the facility item information corresponding to the plurality of facility attribute items chosen.

12. The medical facility matching system according to claim 1, wherein
the at least one processor is further configured to execute the instructions to notify the patient and the at least one medical facility of the matching result, so that decision making on whether to approve the matching result is allowed for the patient and the at least one medical facility.
